(19) United States
(12) Patent Application Publication
(10) Pub. No.: US 2004/0230093 A1
(43) Pub. Date: Nov. 18, 2004

(54) Stimulation aid

(76) Inventor: Ian Peter Marshall, Wellingborough (GB)

Correspondence Address: KNOBLE YOSHIDA & DUNLEAVY, LLC, Eight Penn Center, Suite 1350, 1628 John F. Kennedy Blvd., Philadelphia, PA, 19103, US

(21) Appl. No.: 10/797,643

(22) Filed: Mar. 10, 2004

(30) Foreign Application Priority Data
May 14, 2003 (GB) 0311033.5
Sep. 30, 2003 (GB) 0322923.4

(51) Int. Cl. A61F 5/00

(52) U.S. Cl. 600/38

(57) ABSTRACT

A generally u-shaped implement forming the stimulation aid comprises a handle and a penetrative shaft connected at their respective proximal ends. An inner face of the handle has a series of ribs for clitoral and labial stimulation. A rounded distal end of the penetrative shaft is curved inwardly to stimulate the “G-spot”. The aid is operable manually, using a rocking motion. In some embodiments, it is vibrated and/or pulsed using an electric motor, embedded within the device or inserted into a recess provided for it.

The present invention relates to an aid for stimulation in the form of a curve shaped implement adapted to aid female masturbation. More particularly, but not exclusively, it relates to an implement which may either be a solid body or have one or more cavities for access of a vibration or pulse mechanism, from either batteries or a rechargeable unit. The invention further has utility as an aid for enhancing the marital relationship, such as in relationships wherein the husband suffers from impotence.

It is known to provide traditional penis shaped aids for such purposes. However, it is not always easy for the user to hold such traditional aids and perform other acts.

Other implements have been proposed. For example, U.S. Pat. No. 5,853,362, to Jacobs, discloses a generally J-shaped device, adapted for stimulation of the “G” spot. However, this device provides no external genital stimulation, and in many of its embodiments requires two persons to use it.

U.S. Pat. No. 5,690,603, to Kain, discloses a series of devices intended for use by two persons, one of whom benefits from penetrative stimulation, and the other from external genital stimulation. The devices are not adapted for use by a single user, and are unlikely effectively to stimulate the “G” spot.

U.S. Pat. No. 3,996,930, to Sekulich, discloses a generally V-shaped implement, one leg of which is worn internally and the other leg of which is held in contact with the wearer's body by the pressure of her clothing. This external leg is provided with a series of ribs which may provide clitoral stimulation as the wearer moves around. However, this will produce minimal motion of the internal leg, which is in any case not adapted to stimulate the “G” spot, and the device cannot easily be manipulated to provide additional stimulation.

It is an object of the invention to provide an implement which aids female masturbation by giving simultaneous labial, clitoral, penetrative and “G” spot stimulation from a simple rocking (forwards/backwards) motion of the hand.

According to the present invention there is provided a stimulation aid comprising a generally u-shaped implement having a first handle portion and a second penetrative shaft portion connected together at their respective proximal ends, said shaft portion being curved re-entrantly at its distal end to form a smooth nosed hook and said handle portion having an inner face adapted to act as an outer stimulator zone.

The outer stimulator zone may comprise a plurality of transversely extending ribs.

Alternatively, it may comprise a plurality of protrusions from the plane of the inner face.

Advantageously, an outer face of the handle portion is provided with means to aid manual gripping thereof.

In this case, the outer face may be contoured to provide a palm grip surface.

The shaft portion may taper from its proximal to its distal end.

The shaft portion may be contoured and/or textured along its length for additional stimulation.

The handle portion and the shaft portion may diverge at a general angle in the range 20 to 45°.

Preferably, the angle of divergence is in the region of 25 to 35°.

The aid may comprise a resiliently deformable elastomeric material, preferably latex, jelly, a soft rubber composition, or a silicone composition.

The aid may be provided with means to cause vibration and/or pulsing thereof over at least part of its overall surface.

The vibrator and/or pulsing means may be electrically powered, and may be electronically controlled.

The means to cause vibration and/or pulsing may be adapted to vibrate and/or pulse both the outer stimulator zone of the handle portion and the shaft portion.

Recess means may be provided to receive a vibrator and/or pulsing means separable from the aid.

Optionally, said recess means may extend generally proximally from a distal end of the handle portion.

Warming means may be provided, for example electrically powered warming means, integral of the aid or removably insertable into said recess means.

Optionally, the aid may further comprise a means of anal stimulation.

The means of anal stimulation may comprise a further shaft portion extending from the vaginally penetrative shaft portion at a point adjacent a proximal end thereof.

In an embodiment usable simultaneously by two users, the aid comprises two generally u-shaped implements as described above, each mounted, optionally pivotably, to a respective end of a connecting member.

Embodiments of the invention will now be more particularly described, by way of example, and with reference to the accompanying drawings in which:

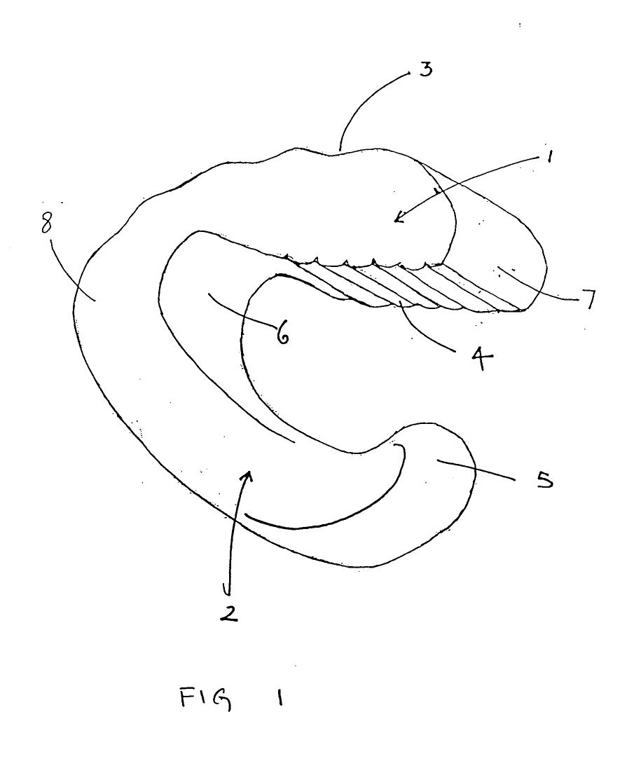

FIG. 1 is a perspective view of a first aid embodying the invention;

FIG. 2 is a schematic cross-sectional view of a handle portion of a second aid embodying the invention;

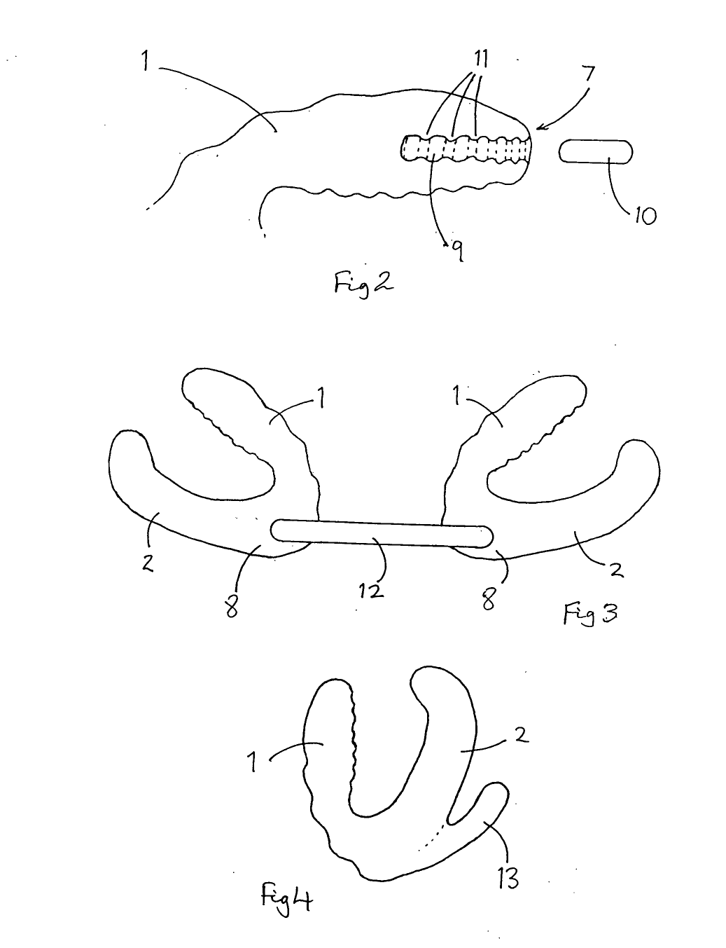

FIG. 3 is an elevation of a third aid embodying the invention; and

FIG. 4 is an elevation of a fourth aid embodying the invention

Referring now to the drawings and to FIG. 1 in particular, a stimulation aid takes the form of a generally U-shaped implement having a handle portion 1 as one leg of the U, and a penetrative shaft portion 2 as the other leg.

The handle portion 1 is provided on its outer surface with contours 3 to aid grip of a palm of a user's hand. An inner surface of a distal end of the handle portion 1 is a zone of transversely extending ribs 4 for stimulation as described below.

The shaft portion 2 tapers slightly from its proximal end to its distal end.

At the distal end, the shaft portion 2 is curved into a re-entrant hook shape 5, the purpose of which is also described below.

In operation, the user may place their palm over the handle portion 1, aided by the contoured outer surface 3 thereof. It is then rocked forwardly and rearwardly. The user's labia and clitoris are both stimulated by the transverse ribs 4 extending across the inner surface of the handle portion 1 when the hand starts the rocking motion. Obviously more or less pressure can be applied by the hand as required. The transverse ribs 4 may be replaced by a series of dimples or protrusions (not shown).

Simultaneously, the user's vaginal entrance and internal vaginal walls are stimulated by the shaft portion 2 of the implement. The shaft portion 2 is tapered from its proximal to its distal end to give extra vaginal entrance stimulation. The distal end of the shaft portion 2 is curved inwardly as a hook 5 so as to stimulate the so-called “G” spot.

The implement may be used either as a simple non-powered aid or it may be in the form of a vibrating and/or pulsing model. In this case both the ribbed zone 4 and the shaft portion 2 will vibrate and/or pulse.

In this field, a widely-used vibrating and/or pulsing device is that known as a “bullet”. This comprises an electrically-driven motor with an eccentrically-mounted load, encapsulated in a generally cylindrical casing with a rounded tip. Such a “bullet” may be powered and controlled remotely along electrical cabling, or may be internally battery-powered. Some versions merely vibrate; others have a range of selectable vibrational and pulsing modes.

The implement may thus be provided with an integral “bullet” or the like, or, preferably, may be provided with a recess 9 as shown in FIG. 2 into which a user may fit a “bullet” 10 of their choice. Conveniently, such a recess 9 will extend proximally into the handle portion 1 from its distal end 7. The particular recess 9 shown is provided with a series of resilient internal ribs 11, so that alternative bullets 10 having a range of different dimensions may be securely held therein. With the correct choice of materials, the vibrations and/or pulses will be transmitted throughout the handle portion and the shaft portion.

The implement may also contain an electrically-powered warming element, applied in the same manner as is the pulsing/vibrating mechanism.

Due to the nature of the design and the fact that all women are of different shapes and sizes the internal recess area 6 of the inner curve provides flexibility of fit, and also facilitates the preferred rocking motion.

The implement is preferably made from a soft rubber, latex, jelly or preferably silicone or other elastomeric compound to give it flexibility. Silicone compounds are particularly suitable, due to their inertness, durability, ease of cleaning and the ready transmission of vibrations therethrough.

The implement may be provided, preferably at its proximal end, with dowel or other fixing means to allow two such implements to be interconnected back to back for simultaneous usage by two people.

Thus, as shown in FIG. 3, two generally U-shaped implements of the form shown in FIG. 1 may each be pivotably mounted, preferably adjacent a junction 8 of the handle portion 1 and shaft portion 2 of each, to respective ends of a connecting rod 12. The connecting rod 12 may be substantially rigid, resiliently flexible or may contain a spring.

In a further embodiment, shown in FIG. 4, the aid is provided with an additional shaft portion 13 extending from adjacent a proximal end of the shaft portion 2. The additional shaft portion 13 is dimensioned and configured to contact or penetrate, in use, the anus of the user, as a further source of stimulation.

1. A stimulation aid comprising a generally u-shaped implement having a first handle portion and a second penetrative shaft portion connected together at their respective proximal ends, said shaft portion being curved re-entrantly at its distal end to form a smooth nosed hook and said handle portion having an inner face adapted to act as an outer stimulator zone.

2. A stimulation aid as claimed in claim 1, wherein the outer stimulator zone comprises a plurality of transversely extending ribs.

3. A stimulation aid as claimed in claim 1, wherein an outer face of the handle portion is provided with contoured means to aid manual gripping thereof.

4. A stimulation aid as claimed in claim 1, wherein the shaft portion tapers from its proximal to its distal end.

5. A stimulation aid as claimed in claim 1, wherein the shaft portion is contoured along its length for additional stimulation.

6. A stimulation aid as claimed in claim 1, wherein the handle portion and the shaft portion diverge at a general angle in the range of 20° to 45°.

7. A stimulation aid as claimed in claim 1, comprising a resiliently deformable elastomeric material.

8. A stimulation aid as claimed in claim 1, provided with means to cause vibrational motion over at least part of the overall surface thereof.

9. A stimulation aid as claimed in claim 8, wherein the means to cause vibrational motion is electrically powered.

10. A stimulation aid as claimed in claim 8, wherein the means to cause vibrational motion is adapted to move both the handle portion and the shaft portion.

11. A stimulation aid as claimed in claim 1, wherein recess means is provided to receive a means to cause vibrational motion separable from the aid.

12. A stimulation aid as claimed in claim 11, wherein the recess means is adapted also to receive a heating means.

13. A stimulation aid as claimed in claim 11, wherein said recess means extends generally proximally from a distal end of the handle portion.

14. A stimulation aid as claimed in claim 11, wherein said recess means is configured to retain a variety of means to cause vibrational motion having a range of different dimensions.

15. A stimulation aid as claimed in claim 1, further comprising a means of anal stimulation.

16. A stimulation aid comprising two generally unshaped implements as claimed in claim 1, each mounted to a respective end of a connecting member.

17. A stimulation aid as claimed in claim 16, wherein each said implement is pivotably mounted to the connecting member.
